(19) United States
(12) Patent Application Publication
(10) Pub. No.: US 2004/0024447 A1
(43) Pub. Date: Feb. 5, 2004

(54) Individual venous valve prosthesis

(76) Inventor: Axel Haverich, Isernhagen (DE)

Correspondence Address: IP Strategies, Suite 500, 1730 North Lynn Street, Arlington, VA, 22209, US

(21) Appl. No.: 10/258,757

(22) PCT Filed: Apr. 27, 2001
(86) PCT No.: PCT/EP01/04796

(30) Foreign Application Priority Data
Apr. 27, 2000 (DE) 100 20 540.2

(51) Int. Cl. A61F 2/06

(52) U.S. Cl. 623/1.24; 623/1.41

(57) ABSTRACT

The invention relates to the use of a recipient-specific transformed synthetic or natural acellularized matrix for the production of an individual venous valve prosthesis.

The invention relates to the use of a recipient-specific transformed synthetic or natural acellularized matrix for the production of an individual venous valve prosthesis.

Vein diseases take a significant place among the civilization diseases. Every year many people, particularly in the so-called industrial nations, are affected with venous insufficiency. Lack of physical exercise contributes to the increasing spreading of this disease as well as nutrition faults and obesity. Chronic venous insufficiency is a problem of significant health-political interest since a considerable part of the adult population is afflicted and a long hospitalization, in individual cases even a disability in the workplace, may be the consequence. Also the danger of pulmonary embolism as a consequence of thrombosis represents a considerable risk. One can hardly deal with chronic venous insufficiency using conservative treatment, surgical stockings and suspensory bandages are used. In advance condition, the venous valves are completely destroyed, i.e., dissolved but also thickened regions occur in the venous valves. The clinical consequences are painful, aesthetically disturbing so-called ulcerated legs or crural ulcer requiring an extremely lengthy treatment. At this stage, only a surgical treatment is possible. Such an operation is part of the field of vessel surgery; the vessel surgeon tries to reconstruct the non-functioning venous valve. Different methods have been tried, for example, the vein reconstruction according to Rutherford and the valvuloplasty according to Kistner. Due to insufficient clinical results, however, none of these methods has been generally accepted up to now.

One drawback of the different surgical reconstruction methods consists in that the valve cannot be restored to the original state; thus, a risk of further deterioration, in particular, crural ulcers, remains.

A problem of the surgical treatment also consists in that the reconstruction has to be performed directly on the patient. Thus, large-scale reconstruction methods involve the drawback of longer operation times and the corresponding risks. It is a further disadvantage that the venous valve is to be fully stressed immediately after the operation; the operation region can be taken care of in no way. Surgery injuries, scars or even small clots at the venous valve lead to a relatively high thrombosis risk that immediately questions the success of these surgical methods.

Thus, it is the problem underlying the invention to open new possibilities to combat the venous insufficiency.

According to the invention, this problem is solved by using a recipient-specific transformed synthetic or natural acellularized matrix for the production of an individual venous valve prosthesis.

It is true that other vessel prostheses are known in principle, for example, heart valve prosthesis are implanted relatively successfully for several years. For venous valves, however, the present valve materials that have prevailed and commercially spread for heart valves are not suitable. It turned out that the thrombosis risk in the field of the smaller dimensioned venous valves is too high due to the different current conditions (little current, small pressure gradient). Up to now, the vessel surgeon did not have a possibility to treat the disease differently than using the above-described insufficient methods.

Surprisingly, it has been found that a recipient-specific transformed synthetic or natural acellularized matrix is suitable for the production of an individual venous valve prosthesis. A great advantage of the invention consists in that the finished, “intact” venous valve prosthesis can be implanted in an altogether shorter operation step, replacing the defective valve. The joints are situated at smooth sections that can be joined in a comparatively uncomplicated way and that lead away from a thrombosis risk in the valve itself.

Since the individual venous valve prosthesis is specifically adapted to the selected recipient, the thrombosis risk can be kept minimal.

By a “recipient-specific transformation”, preferably, a population of the selected matrix by recipient compatible cells is to be understood, in particular, by autologous cells of the prosthesis recipient.

The matrix to be described in more detail below is populated with recipient compatible cells as far as to sufficiently suppress the thrombogenesis of the foreign body “venous valve prosthesis”. The type of the populating cells has an influence on the thrombogenesis as well. Particularly well suited is a population with fibroblasts and endothelium cells and, if need be, also with myofibroblasts.

The matrix used for the recipient-specific transformation can be a synthetic matrix, for example, a bio-polymeric material, a polymeric material common for prosthesis and, in particular, a biodegradable polymeric material. A suitable material, for example, would be a lactid-comprising polymer, preferably, a co-polymer made of lactid and a glycol-compound and, even more preferred, multi-layered polydioxanon.

The matrix can also be a—with respect to the recipient—xenogenic or allogenic matrix out of which or out of the surface of which essentially all natural cells have been removed before the recipient-specific transformation.

Preferably, the base-matrix used for the recipient-specific transformation can be a natural venous valve. The acellularization of xenogenic or allogenic venous valves can be done in a known way, for example, by enzymatic removal of the cells, for example, with trypsin, or by removing and/or killing the cells using chemical and/or mechanical means.

Alternatively, a recipient-specific transformed matrix material can be used for the construction of a venous valve prosthesis. In this case, the venous valve prosthesis can be composed of several synthetic or natural acellularized matrix components. The selected materials are pre-populated before the construction of the venous valve prosthesis and, if necessary, can be covered at the surface after the construction in a further step with a non-thrombogenic material or, additionally, further populated with recipient-specific cells.

Preferably, the venous valve prosthesis comprises at least one valve cusp.

To guarantee a better connection to the vein of the recipient, the venous valve prosthesis in a preferred embodiment may comprise a piece of vein of a specific length: preferably, the venous valve is situated in a piece of vein the length of which above and below the valve region corresponds in each case at least once to the diameter of the vein or the valve cross-section, respectively.

A great advantage of the invention is that the venous valve processed in the above-described manner and, if need be, specifically newly constructed for the recipient, can be prepared, if necessary, in several steps in such a way that the thrombosis risk for the corresponding selected recipient remains as small as possible.

It is a further advantage that the functioning of the venous valve prosthesis can be tested at least in vitro by stretching the venous valve in a corresponding device and perfusing it in a pulsating way with a culture medium or a simple (crystalloid) solution.

1. Use of a recipient-specific transformed synthetic or natural acellularized matrix for the production of an individual venous valve prosthesis.

2. Use according to claim 1, wherein the recipient-specific transformation comprises a population of the matrix with recipient compatible cells, preferably, autologous cells of the prosthesis recipient.

3. Use according to claim 1 or 2 wherein the matrix is a synthetic matrix made of a bio-polymer, a polymer and, in particular, a biodegradable polymer.

4. Use according to claim 1 or 2, wherein essentially all natural calls are removed from a xenogenic or allogenic matrix before the recipient specific transformation.

5. Use according to claim 4, wherein the matrix is a natural venous valve.

6. Use according to one of the claims 1-4, wherein the venous valve prosthesis is constructed out of a recipient-specific transformed matrix material.

7. Use according to claim 6, wherein the venous valve prosthesis comprises at least one valve cusp.

8. Use according to one of the claims 1-7, wherein the venous valve prosthesis comprises a piece of vein of a certain length of preferably at least once the diameter above and below the valve.
